(19) United States
(12) Patent Application Publication
(10) Pub. No.: US 2026/0347927 A1
(43) Pub. Date: Nov. 5, 2026

(54) REFERENCE MATERIAL COMPRISING NUCLEIC ACIDS HAVING A FRAGMENTATION AND NUCLEOSOME PROFILE AND DIAGNOSTIC METHOD

(71) Applicant: SENSID GMBH, Rostock (DE)

(72) Inventors: Björn NOWACK, Seebad-Warnemünde (DE); Angelika LÜKING, Ludwigsfelde (DE); Anna-Maria PIELKA, Rostock (DE)

(21) Appl. No.: 19/487,556

(22) PCT Filed: May 27, 2024
(86) PCT No.: PCT/EP2024/064551

§ 371 Date: Nov. 25, 2025

(30) Foreign Application Priority Data
May 26, 2023 (EP) 23175864.0

(51) Int. Cl. C12Q 1/6869; C12Q 1/44; C12Q 1/6806

(52) U.S. Cl. CPC C12Q 1/6869; C12Q 1/44; C12Q 1/6806; C12Y 301/21001; C12Y 301/31001; C12Q 2600/166

(57) ABSTRACT

The present invention relates to a new reference material comprising nucleic acids with a specific fragmentation pattern, in particular for use in sequencing methods such as polymerase chain reaction (PCR) or next-generation sequencing (NGS), methods for its production and use, and an associated diagnostic. This reference material allows the validation of such methods, including the verification of measurement results, in particular depending on device parameters and sample preparation. Furthermore, the invention comprises an associated diagnostic method for liquid biopsy or cell-free DNA.

The present invention relates to a new reference material comprising nucleic acids with a specific fragmentation pattern, in particular for use in sequencing methods such as polymerase chain reaction (PCR) or next-generation sequencing (NGS), methods for its production and use, and associated diagnostics. This reference material allows the validation of such methods, including the verification of measurement results, in particular depending on device parameters and sample preparation. Furthermore, the invention comprises an associated diagnostic method for liquid biopsy or cell-free DNA.

Current methods for detecting diseases, such as clinical chemical tests in body fluids and tissue samples or imaging techniques, have, despite all the advances made in recent years, usually led to the detection of adverse changes too late. In order to prevent the occurrence of, for example, metastases in tumor diseases, it is necessary to detect malignant changes as early as possible. Furthermore, methods are needed to correctly distinguish diseased cells from healthy cells. Both false-positive and false-negative findings have fatal consequences for those affected. Methods are also needed that allow for a correct prognosis and enable therapy tailored to the individual patient.

The isolation, characterization, and analysis of nucleic acids (ribonucleic acids, RNA, and deoxyribonucleic acids, DNA) from cell materials is usually carried out in the following steps according to the state of the art: sample preparation, extraction, concentration, isolation, purification, reverse transcription if necessary, amplification, and detection.

Due to the powerful amplification of DNA using polymerase chain reaction (PCR), as used in “qPCR” (real-time quantitative polymerase chain reaction), “digital PCR” (dPCR), or various variants of next-generation sequencing (NGS, or parallel sequencing such as massive parallel sequencing), the so-called minimal to non-invasive liquid biopsy has become established in diagnostics, whereby, for example, circulating free DNA in a sample (blood, plasma) is examined. Such circulating free DNA (cfDNA for short) are short double-or single-stranded DNA molecules (50-200 bp) from the fragmentation of genomic DNA from apoptotic and necrotic cells, and are used as biomarkers for the diagnosis of diseases.

In cancer patients and other diseases, the average amount of cfDNA is higher than in healthy subjects (known as ctDNA in tumor diseases). In particular, the cfDNA plasma level in cancer patients in advanced stages is higher than in mild/early stages. ctDNA accounts for 1% of cfDNA (0.001-90% of normal cfDNA, depending on the stage and location of the tumor).

It is also known that cfDNA circulates in the blood or plasma and originates from various cell types, e.g., from various healthy organs/tissues or pathologically altered organs/tissues ( ), e.g., tumors in different anatomical locations. Interestingly, it has also been found that circulating fetal cfDNA (mainly originating from the placenta) and tumor ctDNA are shorter than “background” cfDNA, which is mainly of hematopoietic origin. cfDNA also carries signatures of its association with nucleosomes and chromatin, such as the most common plasma DNA length of 166 bp and fragmentation endpoints that show relationships to nucleosome arrangement.

A nucleosome is the unit consisting of DNA and a histone octamer. The octamer consists of two copies each of the proteins H2A, H2B, H3, and H4. Around this protein complex, 146 or 147 base pairs of DNA are wound as a left-handed superhelix. A chromatosome is the result of the binding of histone H1 to a nucleosome. The chromatosome contains 166 base pairs of DNA. However, in the following, they are collectively referred to as nucleosome(s). In all eukaryotes, DNA is present in a condensed state called chromatin.

While cfDNA spanning the entire genome (both nuclear and mitochondrial) is present in plasma, there are microheterogeneities in cfDNA coverage between different genomic regions. For example, cfDNA abundance and fragment end frequencies differ between open and closed chromatin domains, regions within and between nucleosomes, and specific genetic elements such as transcription factor binding sites. There is evidence that cfDNA has a predisposition to be cleaved at specific genomic regions or elements, resulting in cfDNA exhibiting a higher representation of certain end sequence motifs. Such selectivity of cleavage sites may be related to the sequence motif preferences of the nucleases involved in the process. These relationships are the subject of what is known as “fragmentomics”.

The chromatin structure influences the length and frequency of cfDNA. Open (euchromatin) and closed chromatin regions (heterochromatin) differ in the frequency of nucleosome occurrence and gene expression levels (e.g., due to the presence of transcription factor (TF) binding sites), which in turn can increase or decrease accessibility to nucleases. These variables manifest themselves in the fragmentomic characteristics of cfDNA. The majority of cfDNA is mononucleosomal in length, and nucleosomes influence the length of cfDNA (nucleosome footprint).

Understanding the fragmentation patterns of cfDNA depending on cell type can aid in the identification of pathologies/diseases associated with these tissues or organs. Furthermore, it has recently been shown that fragmentation patterns can differ depending on the tissue of origin (i.e., fetal or maternal, cancerous or non-cancerous) or nucleosomal packaging of the DNA. Therefore, the fragmentation pattern includes not only fragment sizes, but also fragment endpoints and nucleosome footprints. Gene expression is also influenced, i.e., the number of copies or RNA transcripts.

However, one of the major challenges of fragmentomics is pre-analytical aspects such as blood collection and the time sequence with regard to the transport of blood to the laboratory and the processing of blood, in particular sample preparation. In addition, cfDNA extraction and library preparation protocols can preferentially deplete or enrich DNA fragments of certain sizes, so these steps must be carefully planned for meaningful DNA fragmentation analyses. The biological aspects include, for example, the various nucleases involved in fragmentation (e.g., DNA fragmentation factor beta, DNASE1L3, and DNASE1), which leave behind partially overhanging ends with a defined sequence or base background, as well as different sizes.

For these reasons, cfDNA coverage or sequence depth (known as “coverage”) is not uniform but specific, depending on cell type and nucleosome footprint, resulting in specific fragmentation patterns.

Coverage in DNA, especially cfDNA, refers to the number of sequencing reads used to analyze cfDNA. Coverage is usually defined as the average number of reads generated across a specific genome or target region. High coverage means that there are enough reads to obtain accurate information about the underlying DNA or cfDNA sequence. Low coverage, on the other hand, can lead to inaccurate or missing information.

Sufficient coverage is important for identifying rare genetic variants, mutations, or structural changes in DNA or cfDNA. It also enables the precise quantification of specific DNA sequences or the analysis of cell-free DNA fragments in general.

The exact coverage requirement may vary depending on the type of analysis, the question being asked, and the sequencing technology used.

There is therefore a great need for a suitable standard or reference material so that validation or calibration of a cfDNA determination can be performed depending on the coverage requirement of a sequencing method, or related amplicon or non-amplicon methods, such as polymerase chain reaction (PCR) or next-generation sequencing (NGS). Device parameters are also important.

The state of the art describes common laboratory standards.

However, a disadvantage is that these internal laboratory standards must be produced and diluted for the purpose of validating or calibrating a device or method, and no automated process is available for this, so that standardization is usually prone to error. Consequently, the copy number determined by sequencing, cycles, or non-amplicon-based methods may be relatively and absolutely incorrect due to a lack of sufficient standardization. Furthermore, cfDNA is usually not used because it would have to be obtained from blood samples, which may result in insufficient analyzable sequences. The (blood) donors may also be sick or healthy, which could influence and distort the standard.

It is therefore necessary to produce artificial cfDNA as reference material, which has not yet been described in the state of the art.

It is therefore necessary to further improve standardization with the aid of an artificial reference material that corresponds to natural cfDNA, exhibits a natural fragmentation pattern (supra), and satisfies the coverage requirement.

The object of the present invention is therefore to provide an improved method for validating a sequencing method for determining natural cfDNA.

Surprisingly, the inventors found that the use of cfDNA with a specific fragmentation pattern significantly improves validation for such a standard. To this end, it is necessary to take the aforementioned coverage into account so that an improved validation result can be achieved depending on the cell type or organ or tissue. In addition, sufficient analyzable sequences must be available.

The object is therefore solved by a method for validating a sequencing method for determining cfDNA, wherein
i.) a reference material is provided, which is produced with the following steps:
a.) providing a cell culture comprising cells,
b.) treating the cell culture from a.) with an inhibitor for cell division,
c.) isolating nucleic acids comprising nucleosomes,
d.) treating the nucleic acids comprising nucleosomes with nucleases and/or ultrasound,
e.) isolating nucleic acids with a length of 20-750 bp, and
ii.) determining the nucleic acids from e.) using the sequencing method.

The above method is referred to below as the “invention-based validation method.”

Step b.) is particularly advantageous for obtaining or influencing the “coverage” or the desired fragmentation pattern. The cell division inhibitor freezes the state or a selected coverage.

An advantage of the method according to the invention is that the reference material can be produced in as consistent a quality as possible across all production cycles. This is particularly difficult because each cell in that cell culture is at a different stage of the cell cycle. As a result, one cell does not have the same DNA content at the moment of cell harvest as another cell. Similarly, the distribution of heterochromatin and euchromatin varies between cells, affecting the transcriptome and methylation pattern. To counteract this, the cells can be treated with a cell division inhibitor, which aims to transfer all cells to the same cell cycle at the time of harvest.

In order to advantageously obtain all DNA packed onto nucleosomes in cell culture, the cells are preferably treated with a mitosis inhibitor, preferably colcemide (also known as colchicine), so that mitosis is stopped in metaphase. This is advantageous in order to obtain the highest possible coverage. Furthermore, uniform production of the reference material across each batch is advantageously achieved, as the cells can always be harvested in the same cell cycle. When colchicine is added to cultured eukaryotic cells, the cells enter mitosis and arrest in metaphase with condensed chromosomes.

Therefore, for the purposes of this invention, such cell division inhibitors, in particular mitosis inhibitors or checkpoint inhibitors, selected in particular from the group consisting of colcemid (CAS number: 477-30-5), benomyl (CAS number: 17804-35-2), nocodazole (CAS number: 31430-18-9), paclitaxel (CAS number: 33069-62-4), vincristine (CAS number: 57-22-7), vinblastine (CAS number: 865-21-4), 8-chloroadenosine (CAS number: 34408-14-5), WYE 687 dihydrochloride (CAS number: 1702364-87-1), AZD 5438 (CAS number: 602306-29-6), Pladienolide B (CAS number: 445493-23-2), Temsirolimus (CAS number: 162635-04-3), Tosyl-L-Arginine Methyl Ester (TAME) (CAS number: 1784-03-8), YC 1 (CAS number: 170632-47-0), Indisulam (CAS number: 165668-41-7).

Furthermore, nucleases according to the invention are used in accordance with step d.), selected from the group MNase (EC number: 3.1.31.1), DNase1L3 (EC number: 3.1.21.1), DNase I (EC number: 3.1.21.1).

Following enzymatic digestion, the DNA can be purified and, optionally, an end repair reaction can be performed using Klenow/T4 DNA polymerase (EC number: 2.7.7.7).

Alternatively, ultrasound can be used according to step d.) to obtain nucleic acids with a length of 20-750 bp.

However, the use of nucleases according to step d. ) is preferred.

For the purposes of this invention, “reference material” means a nucleic acid produced in steps a.)-e.) with a length of 20-750 bp, in particular 50-500 bp, in particular 20-200 bp. These produced nucleic acids are “artificial cfDNA” within the meaning of this invention, or are cfDNA or represent cfDNA or a totality of cfDNA.

In a further embodiment of the invention, the cell culture comprising cells according to step a) may comprise one or more specific cell types, in particular those capable of mitotic or meiotic cell division, selected from the group of human cells, mammalian cells, eukaryotic cells, somatic cells, stem cells, B cells, T cells, natural killer cells, diseased or healthy cells, tumor cells, cells from a tissue or organ. Also included are monoclonal cell cultures of immortal eukaryotic cells, in particular those derived from polyclonal cultures by isolation. Immortalized cells can be created by infection with viruses, e.g., infection of lymphocytes with Eppstein Barr virus, or by culturing tumor cells that have been removed from a diseased mammal. This initially produces a mixed culture of many individual cells (known as a polyclonal culture). Isolation techniques can be used to create new cell cultures from individual cells (so-called monoclonal cultures). Isolation eliminates the e differences that can occur at the DNA level between cells in a polyclonal culture. This makes the material particularly clean and allows it to be used for the validation of highly sensitive methods in particular. Such monoclonal cell cultures or immortalized cells are therefore particularly preferred due to the identity or homogeneity of the cells. Consequently, the reference material obtained according to the invention has few impurities.

The method according to the invention advantageously allows the provision of a reference material that reproduces the typical and specific fragmentation pattern.

For the purposes of this invention, “sequencing method” means any suitable sequencing method for sequencing nucleic acids, DNA, in particular cfDNA, in particular according to the Sanger method or so-called 2nd generation or amplicon or non-amplicon methods. Amplikon or non-Amplikon methods include pyrosequencing, sequencing by hybridization, ion semiconductor DNA sequencing systems, nanopore sequencing, next-generation sequencing (NGS), sequencing-by-synthesis (SBS), polymerase chain reaction (PCR), qPCR (real-time quantitative polymerase chain reaction), and digital PCR (dPCR).

For the purposes of this invention, amplicon methods are those that require an amplicon for sequencing. Here are some common sequencing methods used for amplicons:
i.) Sanger sequencing: This classic sequencing method is based on the use of terminator nucleotides to determine the DNA sequence. Sanger sequencing is often used for the analysis of short amplicons, typically up to a few hundred base pairs.
ii.) Next-generation sequencing (NGS): NGS technologies such as Illumina sequencing or Ion Torrent sequencing enable the sequencing of many amplicons simultaneously. With NGS, hundreds of thousands to millions of reads can be generated, resulting in a high throughput rate. NGS is suitable for sequencing amplicons of various lengths, from a few hundred to several thousand base pairs.
iii.) PacBio sequencing: Pacific Biosciences (PacBio) single-molecule sequencing technology enables the sequencing of long amplicons. It offers a very long read length and is therefore well suited for sequencing amplicons ranging from several thousand to tens of thousands of base pairs. iv.) Oxford Nanopore sequencing: Oxford Nanopore sequencing also enables the sequencing of long amplicons. The technology is based on performing sequencing in real time by passing DNA molecules through a nanopore. It offers a long read length and is portable.

For the purposes of this invention, non-amplicon methods refer to sequencing methods that do not require amplicons.

For the purposes of this invention, validation means that nucleic acids (or reference material) produced according to the method of the invention a.) to e.) can be determined by means of sequencing using a validation sample. This positive detection may depend on device parameters on the one hand and on the performance of the sequencing method on the other. The validation sample can be provided by means of a kit.

If the validation sample is not found during the sequencing process, sensitivity is not given or the detection limit is too high, meaning that detection cannot take place.

Another special embodiment of the invention relates to a method for diagnosing or prognosticing a disease, wherein cfDNA is determined, in particular for assessing the severity and for evaluating the course of therapy, for examining minimal residual disease (MRD) and therapy decisions, wherein calibration is performed using the validation method according to the invention.

The method according to the invention allows the detection limit of cfDNA to be validated in a particularly advantageous manner. A particularly advantageous application therefore relates to the validation of devices for performing, in particular, a polymerase chain reaction (PCR) or next-generation sequencing (NGS), whereby the method according to the invention is carried out.

In this way, it is particularly advantageous to introduce a precisely specified amount of DNA material or concentration of the reference material according to the invention into a sample or comparison sample, whereby this sample largely simulates a sample and is highly suitable for performing diagnostics.

The term “diagnosis” encompasses medical diagnostics and related examinations, in particular in vitro diagnostics and laboratory diagnostics. Preferably, the statement about a disease or condition of a patient can be made based on the determination of cfDNA.

In the context of this invention, “patient” refers to any test subject.

The invention is explained below using examples and figures. However, the invention is not limited to the examples and figures, but is fundamentally universally applicable.

Example 1: Validation of a diagnostic method for detecting minimal residual disease (MRD) or monitoring/follow-up examinations in cancer.

In one study, fragmented DNA was used in an NGS procedure to validate this. For comparison, various other materials of non-natural origin were used in an attempt to validate the system. Isolated cell-free DNA from a healthy donor was used as a normal control. It was found that the DNA produced as described in the method could be used to validate the system, while other materials were unsuitable due to their dissimilarity to the patient material.

In particular, the fragment size distribution and conversion efficiency were examined. Conversion efficiency refers to the successful processing of the DNA fragments in order to prepare them for sequencing.

In order for the validation of a system, in particular for the detection of MRD, to be successful, the material used for validation must be very similar to the patient material and particularly clean (have a low error rate).

It was found that both the conversion efficiency and the size distribution of the material, when produced according to the invention, are so similar to the patient material that the system can be successfully validated.

In contrast, all other materials tested in the same experiment did not behave in the same way as the patient material.

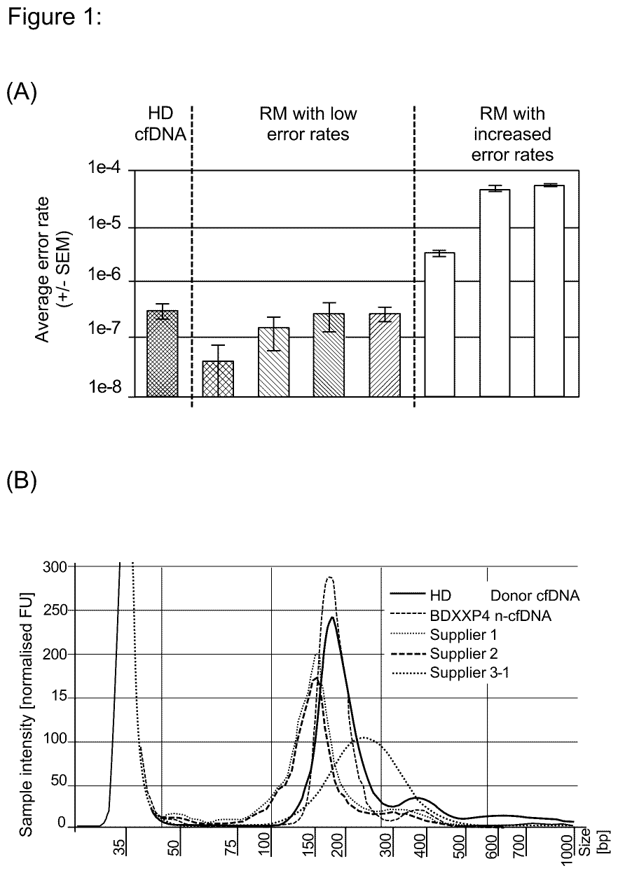

FIG. 1:
A) The mean error rate was determined for various cfDNA-like control materials (RM) and cfDNA from healthy donors (black bar, HD cfDNA) using the MRD test (supra). The samples were sequenced at comparable target depths of ~100,000×.
B) The cfDNA produced using the method described here (see line for BDXXP4 n-cfDNA) has a size distribution that is most similar to that of healthy patient cfDNA (black solid line, healthy donor (HD) cfDNA).

Example 2: Validation of a diagnostic method for detecting a genetic mutation in the context of a pregnancy examination.

The technologies mentioned, such as PCR and NGS methods, can also be used to detect diseases in an unborn child in the mother's blood. This can be done by means of a liquid biopsy and subsequent DNA extraction from the blood. In addition to maternal DNA, the extracted DNA often contains a proportion of fetal DNA. The proportion of fetal DNA is often in the range of 1-10%. Fetal DNA differs from maternal DNA in terms of size, among other things. Fetal DNA is on average smaller than maternal DNA. The present invention therefore allows technology that examines extracted DNA from mothers to be validated. This is done, for example, by mixing two differently fragmented DNA strands in a ratio that is similar to the natural ratio.

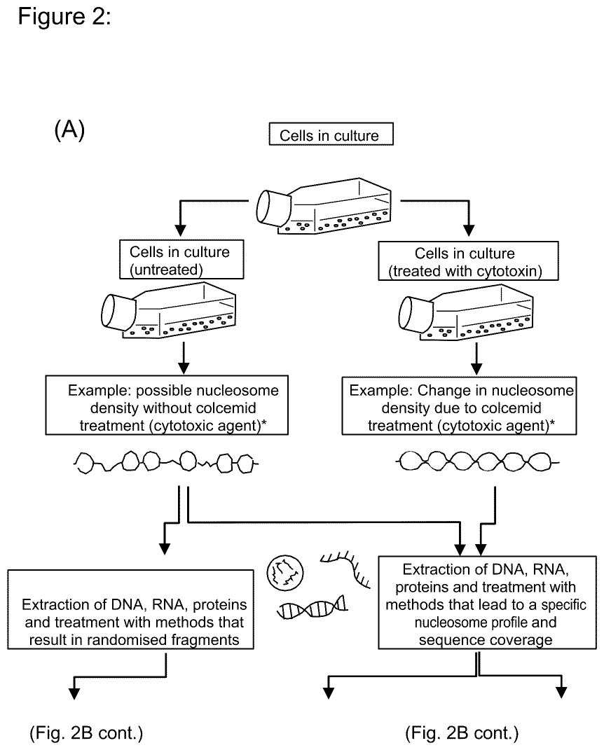

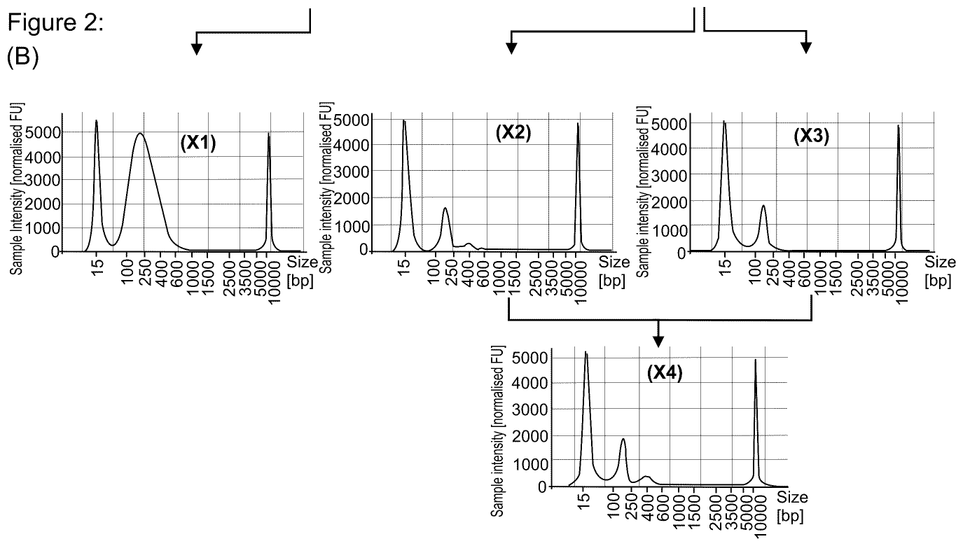

In the present example, an experiment was conducted to investigate whether a mixture of two DNA fragments of different lengths (see FIG. 2B X2 and X3) can be distinguished from each other in a PCR procedure. It was found that the individual DNA fragments in the mixture (see FIG. 2B X4) can be distinguished from each other by PCR.

FIG. 2:

FIG. 2 shows:
(A) Possible differences in nucleosome density when treated with one or no cytotoxins;
(B) Possible fragmentation profiles after treatment with methods that lead to randomized DNA fragments (X1, e.g., ultrasound), or treatment of DNA with nucleases that lead to cfDNA fragmentation profiles, as in cfDNA from healthy individuals (X2) and sick individuals (X3). X4=superimposition of X2 and X3.
   *Other cytotoxins can produce a different nucleosome density. This influences the coverage profile after treatment with a corresponding nuclease.

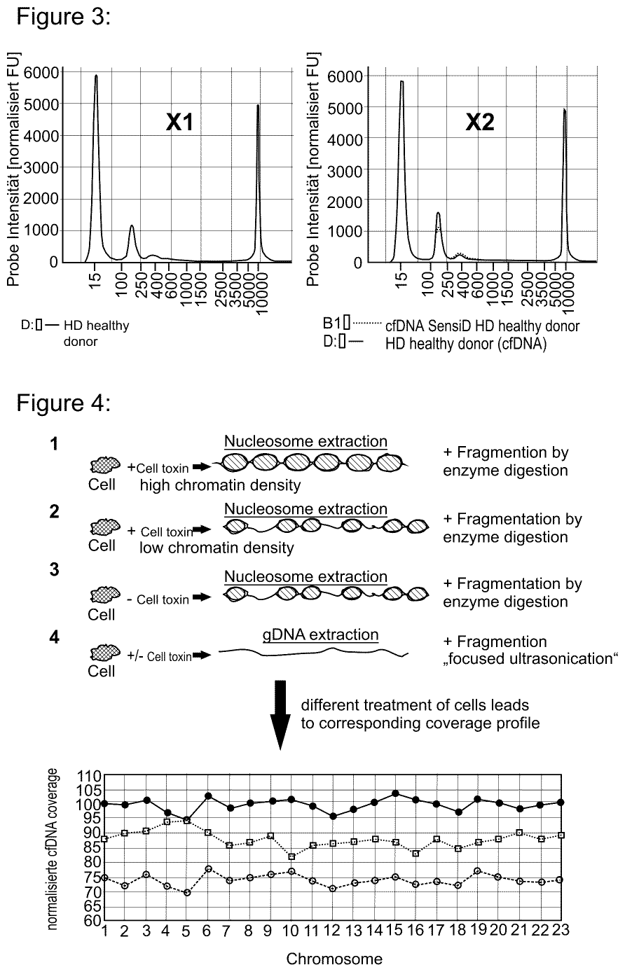

FIG. 3:

FIG. 3 shows (X1) the normal fragment length distribution in humans and (X2) superimposition with DNA produced using the method according to the invention.

FIG. 4:

Possible methods for producing fragmented DNA.

DNA can be produced either by (1,2) nuclease digestion of DNA from cells treated with a cytotoxin, or (3) by nuclease digestion of DNA from cells not treated with a cytotoxin, or by (4) methods that result in randomized fragments after gDNA extraction. The method described in (1,2) can be carried out using cytotoxins that cause arrest in different cell cycle phases. If cytotoxins that arrest the cell cycle in a phase that leads to high chromatin density are used, as in the method described in (1), this results in high coverage. If cell toxins are used that arrest the cell cycle in a phase that leads to low chromatin density, as in the method described in (2), this results in low coverage. (1) and (2) have the advantage that the same coverage is achieved between different production batches with respect to defined sequences, whereas this is not achieved by methods as described in (3).

Consequently, better reproducibility in production is achieved through the use of cell toxins according to the invention, and therefore a defined coverage is achieved. The particular advantage of (1) in the figure is that higher and more uniform coverage is achieved across the entire sequence examined. Through the use of colchicine, for example, most sequences are wrapped around histones (maximally condensed). This results in very high coverage across the entire genome.

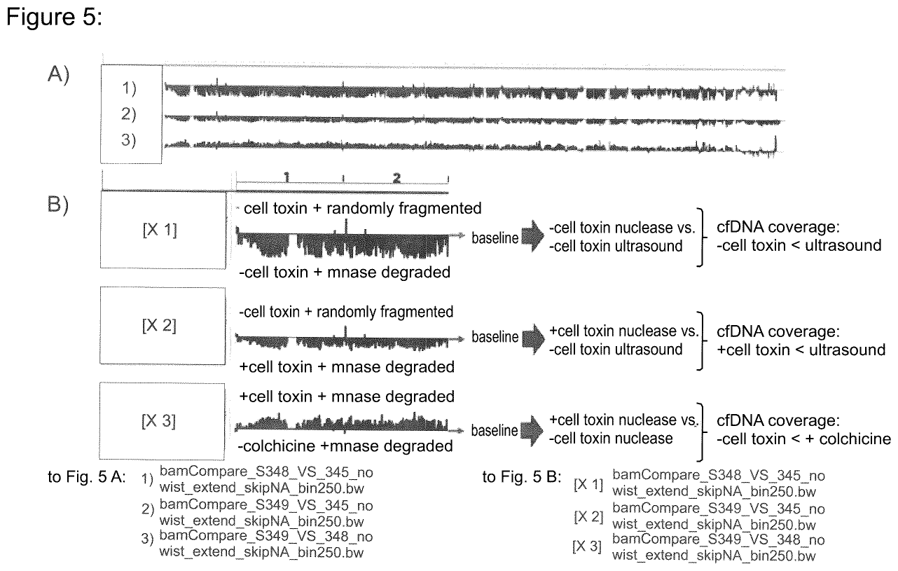

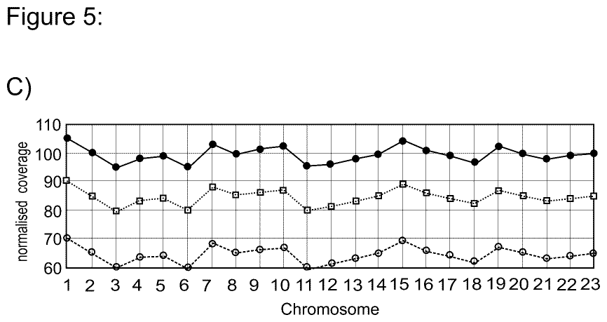

FIG. 5:

FIG. 5 shows a coverage comparison between cells treated with cell cycle inhibitor (+cytotoxin) and cells not treated with cell cycle inhibitor (−cytotoxin) after the DNA was fragmented in different ways.
(A) Coverage ratio between the individual production methods in the whole genome overview. Whole exome sequencing (WES) data was collected. The comparison shows differences in coverage between DNA from cells treated with a cytotoxin, which leads to high chromatin density (+cytotoxin), and cells treated without cytotoxin (-cytotoxin) and subsequently digested with Mnase. In comparison, coverage is highest for randomly fragmented DNA.
(B) Detailed comparison of chromosomes 1 and 2. [X1] compares the coverage between cells not treated with cytotoxin (−cytotoxin+nuclease) whose gDNA was randomly fragmented with nuclease-digested DNA from cells that were not treated with cytotoxin. The coverage is lower in cells that were not treated with cytotoxin ( ), as shown here by the downward deviation from the baseline. [X2] compares the coverage between cells that were not treated with cytotoxin and whose gDNA was randomly fragmented with nuclease-digested DNA from cells that were treated with cytotoxin (+cytotoxin+nuclease). Coverage is lower in cells that have not been treated with cytotoxin, shown here by the downward deviation from the baseline. [X3] compares the coverage between cells not treated with cytotoxin whose DNA was digested with nuclease (−cytotoxin+nuclease) and nuclease-digested DNA from cells treated with cytotoxin (+cytotoxin+nuclease). Coverage is higher in cells treated with cytotoxin, shown here by the upward deviation from the baseline.
(C) Simplified Representation of Coverage Relative to Each other (blue=randomly fragmented DNA, orange=+cytotoxin+Mnase digestion, gray=−cytotoxin+Mnase digestion)

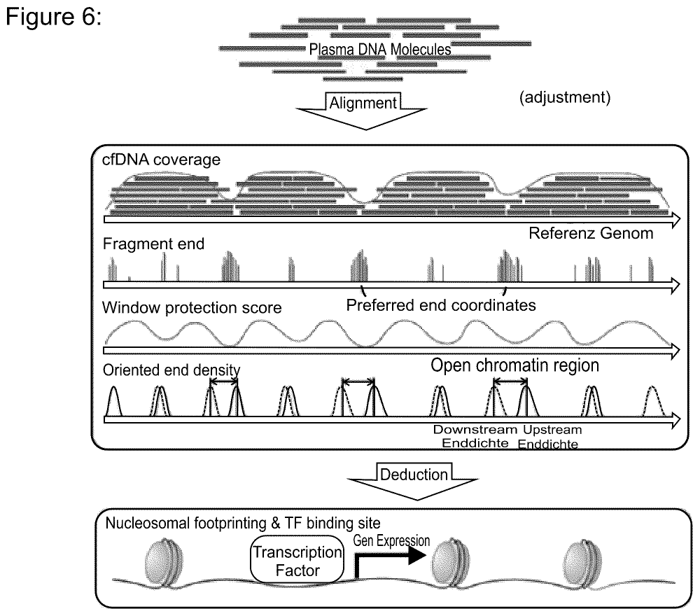

FIG. 6 from (8) shows the relationship between cfDNA coverage, preferred end coordinates, window protection score, and open chromatin regions.

When an Alignment (also Known As Sequence Alignment; Sequence alignment refers to the methodical comparison of two or more nucleotide or amino acid sequences in linear order) is performed on pools of fragmented DNA (in this case, plasma DNA molecules), their ends accumulate at specific genomic locations, known as preferred end coordinates, which can vary between DNA molecules originating from different tissues. The “window protection score” (calculated as the number of complete fragments minus the number of fragment endpoints within a specific “window”) provides information about DNA protection against nuclease digestion, from which nucleosome positioning can be inferred. Genomic coverage reflects the chromatin structure of the source tissue.

### LITERATURE

1. Snyder MW, Kircher M, Hill AJ, Daza RM, Shendure J.: Cell-free DNA comprises an in vivo nucleosome footprint that informs its tissues-of-origin. Cell 2016;164:57-68
2. Jiang P, Sun K, Tong YK, Cheng SH, Cheng THT, Heung MMS, et al.: Preferred end coordinates and somatic variants as signatures of circulating tumor DNA associated with hepatocellular carcinoma. Proc Natl Acad Sci USA 2018;115:E10925-33.
3. Ivanov M, Baranova A, Butler T, Spellman P, Mileyko V.: Non-random fragmentation patterns in circulating cell-free DNA reflect epigenetic regulation. BMC Genomics 2015 ;16:S1
4. Hardham, A. R., Gunning, B.E.S. Some effects of colchicine on microtubules and cell division in roots of Azolla pinnata. Protoplasma 102, 31-51 (1980).
5. Dane, F., Dalgiç, Ö. The Effects of Fungicide Benomyl (Benlate) on Growth and Mitosis in Onion (Allium cepa L.) Root Apical Meristem. BIOLOGIA FUTURA 56, 119-128 (2005)
6. Sun K, Jiang P, Cheng SH, Cheng THT, Wong J, Wong VWS, Ng SSM, Ma BBY, Leung TY, Chan SL, Mok TSK, Lai PBS, Chan HLY, Sun H, Chan KCA, Chiu RWK, Lo YMD. Orientation-aware plasma cell-free DNA fragmentation analysis in open chromatin regions informs tissue of origin. Genome Res. 2019 Mar;29(3):418-427. doi: 10.1101/gr.242719.118. PMID: 30808726; PMCID: PMC 6396422.
7. Sims, David; Sudbery, Ian; Ilott, Nicholas E.; Heger, Andreas; Ponting, Chris P. (2014). “Sequencing depth and Coverage: key considerations in genomic analyses”. Nature Reviews Genetics. 15 (2): 121-132. Doi:10.1038/nrg3642. PMID 24434847. S2CID 13325739.
8. Y. M. Dennis Lo, Diana S. C. Han, Peiyong Jiang and Rossa W. K. Chiu (2021). “Epigenetics, fragmentomics, and topology of cell-free DNA in liquid biopsies” Science; Vol 372, Issue 6538; DOI: 10.1126/science.aaw3616

1-12. (canceled)

13. Method for validating a sequencing method for the determination of cfDNA, characterized in that
i) a reference material is provided, which is produced with the following steps:
a.) providing a cell culture comprising cells,
b.) treating the cell culture from a.) with an inhibitor for cell division,
c.) isolating nucleic acids comprising nucleosomes
d.) treating the nucleic acids comprising nucleosomes with nucleases or ultrasound,
e.) isolating nucleic acids with a length of 20-750 bp, and
ii) determining the nucleic acids from e.) using a sequencing method.

14. Method for validating a sequencing method for determining cfDNA according to claim 13, wherein the cell culture comprising cells are eukaryotic cells, mammalian cells, or human cells.

15. Method for validating a sequencing method for the determination of cfDNA according to claim 13, wherein the cell culture comprising eukaryotic cells is one or more specific cell types selected from the group consisting of somatic cells, stem cells, B cells, T cells, natural killer cells, diseased or healthy cells, tumor cells, polyclonal cells, monoclonal cells, immortalized cells.

16. Method for validating a sequencing method for the determination of cfDNA according to claim 13, wherein the cell division inhibitor is selected from the group consisting of mitosis inhibitors, checkpoint inhibitors, selected from the group consisting of colcemid (CAS number: 477-30-5), benomyl (CAS number: 17804-35-2), nocodazole (CAS number: 31430-18-9), paclitaxel (CAS number: 33069-62-4), vincristine (CAS number: 57-22-7), vinblastine (CAS number: 865-21-4), 8-chloroadenosine (CAS number: 34408-14-5), WYE 687 dihydrochloride (CAS number: 1702364-87-1), AZD 5438 (CAS number: 602306-29-6), Pladienolide B (CAS number: 445493-23-2), Temsirolimus (CAS number: 162635-04-3), Tosyl-L-Arginine Methyl Ester (TAME) (CAS number: 1784-03-8), YC 1 (CAS number: 170632-47-0), Indisulam (CAS number: 165668-41-7).

17. Method for validating a sequencing method for determining cfDNA according to claim 13, wherein the nuclease is selected from the group MNase (EC number: 3.1.31.1), DNase1L3 (EC number: 3.1.21.1), DNase I (EC number: 3.1.21.1).

18. Method for validating a sequencing method for determining cfDNA according to claim 13, characterized in that a validation calibration curve is obtained.

19. Method for validating a sequencing method for the determination of cfDNA according to claim 13, characterized in that a sequencing or method selected from the group pyrosequencing, sequencing by hybridization, ion semiconductor DNA sequencing system, nanopore sequencing, next generation sequencing (NGS), sequencing-by-synthesis (SBS), polymerase chain reaction (PCR), qPCR (real-time quantitative polymerase chain reaction), digital droplet PCR (ddPCR) is performed.

20. Method for validating a sequencing method for the determination of cfDNA according to claim 13, characterized in that the isolated nucleic acids according to claim 13 e.) form a validation sample.

21. Method for diagnosing a disease, wherein cfDNA is determined, characterized in that calibration or validation is performed using a method according to claim 13.

22. Method for detecting minimal residual disease, wherein cfDNA is determined, characterized in that calibration or validation is performed using a method according to claim 13.

23. Use of a kit for carrying out a method according to claim 13.
